(19) United States
(12) Patent Application Publication
(10) Pub. No.: US 2022/0315882 A1
(43) Pub. Date: Oct. 6, 2022

(54) EQUAL ENERGY DEFORMATION COMPOSITE FOUNDATION USING MICROORGANISMS TO SOLIDIFY AGGREGATE AND THE CONSTRUCTION METHOD THEREOF

(71) Applicant: HAINAN UNIVERSITY, Haikou, Hainan (CN)

(72) Inventors: Jun Hu, Haikou, Hainan (CN); Hui Zeng, Haikou, Hainan (CN); Dongling Zeng, Haikou, Hainan (CN); Zhixin Wang, Liyang, Jiangsu (CN); Lin Jia, Haikou, Hainan (CN)

(21) Appl. No.: 17/310,779

(22) PCT Filed: May 21, 2021
(86) PCT No.: PCT/CN2021/095280

§ 371 Date: Aug. 24, 2021

(30) Foreign Application Priority Data
Oct. 12, 2020 (CN) 202011081923.6

(51) Int. Cl. C12N 1/20; C09K 17/40; E02D 3/08

(52) U.S. Cl. CPC C12N 1/20; C09K 17/40; E02D 3/08

(57) ABSTRACT

The present invention discloses an equal energy deformation composite foundation using microorganism to solidify aggregate and a construction method thereof, the composite foundation comprises a pile body and a cushion layer, wherein the pile body is provided with several piles, the cushion layer is arranged at the top of the pile body, the pile body is connected into an integral structure through the cushion layer, and the pile body and the cushion layer are filled with aggregate solidified by microorganism. The method comprises the following steps: step 1, leveling the site; Step 2, construction preparation; Step 3, the pile driver in place; Step 4, forming a hole by hammering; Step 5, filling aggregate into the hole; Step 6, repeating the work of step 5; Step 7, forming an equal energy deformation compaction pile using microorganism to solidify aggregate; Step 8, moving to the next pile; Step 9, tamping the ground; Step 10, until the cushion is flush with the surface. Beneficial effects: using local materials, turning waste into wealth, being environmental friendly, saving project cost and conforming to the concept of green development.

### FIELD OF THE INVENTION

The present invention relates to a composite foundation and a construction method thereof, in particular to an equal energy deformation composite foundation using microorganism to solidify aggregate and a construction method thereof.

### BACKGROUND OF THE INVENTION

At present, large-scale engineering construction is being carried out on the vast islands and reefs far away from the mainland. However, the islands and reefs are far away from the mainland, sand and gravel and fresh water resources are scarce, traffic is inconvenient; if sand and fresh water are shipped from the distant mainland, according to the shipping cost of bulk goods, the shipping cost per cubic meter of sand and fresh water is ¥1600-1900, and considering the freight of cement and mineral admixtures, the raw material cost per cubic meter of concrete is as high as ¥2600-2800, which greatly increases the project cost, and it is difficult to guarantee the construction period due to the influence of wind, waves and the like.

At present, the calcareous sand obtained from excavated lagoons and waterways is used as a material to build artificial islands in the island building project of related islands and reefs. Calcareous sand is mainly formed from the remains of marine organisms such as corals, shells and algae through physical, chemical and biological action; its main component is carbonate sediments, and its CaCO₃ content is over 95%. The environment of artificial hydraulic filling islands and reefs is complex, including long-term corrosion of seawater, complex geological structure characteristics, various extreme environmental conditions and the like. Untreated calcareous sand foundation which may incur excessive settlement, uneven settlement and sandy soil liquefaction under complex geological environment, can not be directly used in engineering construction, so it is necessary to reinforce the reef foundation of hydraulic filling islands.

Traditional reinforcement methods, such as cement reinforcement and chemical grouting, may cause many problems when used in marine environment, for example, cement will be corroded by seawater, and chemical grouting reinforcement may cause environmental pollution and the like. Microbial Induced Calcium Carbonate Precipitation (MICP) is a new reinforcement technology proposed in recent years; it utilizes the metabolic activity of microorganisms to promote of calcium carbonate crystallization between sand particles, cementing sand particles and improving sand strength. This technology has little impact on the environment, and the cementing calcium carbonate is durable in marine environment.

Although the islands and reefs lack natural sand and stone and fresh water resources, coral and seawater resources are abundant. Coral, with light weight, numerous holes and strong water-absorbing ability, is a natural lightweight aggregate; its main mineral components are aragonite and calcite, and its CaCO₃ content is over 96%, so it is a high-quality gravel aggregate. Seawater, rich in various cations and chloride ions, could be used as cementing liquid (also called reaction liquid) in microbial solidification.

With the continuous improvement of China's comprehensive strength, construction projects are becoming higher, more complex and in larger scale; the demolition of old buildings and the emergence of new ones will produce a large amount of construction waste. According to relevant statistics, the total amount of construction waste produced by demolishing old buildings and constructing new buildings in China is over 150 million tons every year, wherein the waste concrete and waste blocks and the like account for about ⅓ of the total amount of construction waste. Due to the immaturity of solid waste treatment technology, these construction wastes are mainly transported to the periphery of cities for disposal, which wastes resources and affects the environment, violating the requirements of sustainable development strategy and green development in China. These discarded concrete and blocks not only occupy a lot of land resources, but also cause serious damage to the ecological environment, which seriously restricts social progress and harmonious development. In recent years, people have gradually realized the seriousness of the consequences; how to treat and utilize discarded concrete and blocks in construction waste, and make them a recycling and profitable resource, has become one of the hot spots and frontier issues in academic and engineering circles.

On the one hand, waste concrete and waste blocks in construction waste are produced in demolishing the existing old building structures and constructing new building projects, and in building damage caused by earthquakes, fires and other disasters. If we can turn waste concrete and other construction waste into useful materials, the greatest advantage is to protect the ecological environment and reduce the exploitation of natural sand and gravel resources, so as to achieve the goal of sustainable development.

Carrier pile composite foundation is a composite foundation treatment technology, which takes plain carrier pile construction as reinforcement to realize pile-soil joint stress; this technology can be applied to both building foundation treatment (rigid foundation) and subgrade treatment (flexible foundation). Carrier pile composite foundation in building includes: rigid foundation, cushion, plain carrier pile and soil between piles; Carrier pile composite foundation in subgrade includes geotextile, cushion, plain carrier pile and soil between piles. By adjusting the displacement of pile and soil at the top of pile under the help of the cushion, the common stress of pile and soil can be realized, which not only exerts the bearing capacity of single pile of carrier pile, but also rationally utilizes the soil between piles. In order to give full play to the higher bearing capacity of single pile, the top of the pile is expanded in a certain depth range, and more loads on the upper part are transferred to the deep soil through the single pile.

Compared with traditional CFG pile composite foundation, the bearing capacity of composite foundation is higher than that of CFG pile due to the high bearing capacity of single pile of carrier pile; the top of the pile is expanded within a certain range, and more loads are transferred to the soil at the end of the pile through the pile; the bearing capacity and modulus of the soil at the end of the pile are higher than those of the shallow soil, which effectively reduces the foundation settlement; Compared with CFG pile, the cost is reduced by 10-30%.

Microbial Induced Calcium Carbonate Precipitation (MICP) is a new soil reinforcement technology proposed in recent years. It utilizes the metabolic activity of microorganisms to promote precipitation of calcium carbonate crystallization between sand particles, cementing soil particles and improving soil strength; this technology has little impact on the environment, and the cemented soil is durable.

How to combine the treatment technology of solid waste such as construction waste with the treatment technology of carrier pile composite foundation and microbial induced calcium carbonate precipitation (MICP) technology to provide a construction method of equal energy deformation composite foundation with microbial solidification of construction waste filler is a problem to be solved at present.

### SUMMARY OF THE INVENTION

The main purpose of the present invention is to solve the problem of using microorganism to solidify coral aggregate in order to reinforce the foundation of hydraulic filling islands and reefs;

Another object of the present invention is how to combine the treatment technology for solid waste such as construction waste with the carrier pile composite foundation treatment technology and the microbial induced calcium carbonate precipitation (MICP) technology, to provide an equal energy deformation composite foundation using microorganism to solidify construction waste filler and a construction method thereof.

The present invention provides an equal energy deformation composite foundation using microorganism to solidify aggregate and a construction method thereof in order to achieve the above purpose and solve the above problems.

The equal energy deformation composite foundation using microorganism to solidify aggregate provided by the present invention comprises a pile body and a cushion layer, wherein a plurality of piles are arranged in the pile body, the cushion layer is arranged at the top of the pile body, the pile body is connected into a whole structure by the cushion layer, and the aggregate solidified by microorganism is filled in the pile body and the cushion layer.

The microorganism is Bacillus pasturii.

The aggregate is coral aggregate or construction waste, the coral aggregate is composed of coarse aggregate and fine aggregate, the coarse aggregate is coral gravel, and the fine aggregate is coral sand, namely calcareous sand; construction waste includes concrete block, crushed stone, plain soil, metal, brick, tile and gypsum; after treatment of screening, rolling and crushing, the particle size of construction waste is ≤30 mm.

A construction method of equal energy deformation composite foundation using microorganism to solidify aggregate provided by the present invention, the method is as follows:

Step 1, cleaning and leveling the site;

Step 2, construction preparation: carrying out construction setting-out and line inspection; checking and adjusting construction equipment;

Step 3, the pile driver in place: the center of the heavy hammer is aligned with the center of the pile position;

Step 4, forming a hole by hammering: lifting a heavy hammer at a certain height to make it fall freely and impact the foundation soil to form a hole to a design or controlled depth;

Step 5, filling aggregate into the hole, pouring microbial solidification liquid with the same volume as the aggregate, wherein the microbial solidification liquid is mainly composed of bacteria liquid and cementing liquid, lifting a heavy hammer at a certain height, and ramming the filler repeatedly;

Step 6, under the action of standard ramming energy, the last penetration amount of the heavy hammer is measured, and when it is no greater than the design requirement, the work of step 5 is repeated;

Step 7, repeating the steps 5 and the step 6, ramming and filling the pile hole to the ground, and finally forming an energy deformation compaction pile using microorganism to solidify aggregate in the foundation;

Step 8, after one pile is formed, the equipment is moved to the next pile;

Step 9, after all piles are formed, the ground of the construction area is tamped by using a plate compactor;

Step 10, back-filling a layer of aggregate and microbial solidification liquid with the same volume as aggregate on the tamped ground, the back-filling elevation is higher than the ground surface by more than 0.2 m, then lifting the plate compactor to a certain height, and ramming the aggregate cushion after microbial solidification on the ground repeatedly until the cushion is flush with the ground surface.

The aggregate is coral aggregate or construction waste, the coral aggregate is composed of coarse aggregate and fine aggregate, the coarse aggregate is coral gravel, and the fine aggregate is coral sand, namely calcareous sand; construction waste includes concrete block, crushed stone, plain soil, metal, brick, tile and gypsum; after treatment of screening, rolling and crushing, the particle size of construction waste is ≤30 mm.

The microorganism is Bacillus pasturii and the microorganism solution is obtained by means of indoor sterile culture, centrifugal concentration, low temperature transportation and on-site expanded culture, the specific method is as follows:

Step 1, indoor culture: every liter of culture medium contains tryptone 15.0 g, Soybean peptone 5.0 g, sodium chloride 5.0 g, putting the prepared nutrient solution in an autoclave, autoclaving at 121° C. for 20 min, and then cool it down in a sterile operation table; in order to avoid the decomposition of urea at high temperature, 20 g urea is added into the bottle when the temperature drops to room temperature, and the pH is adjusted to 7.3; after microbial inoculation, it is incubated at constantly 30° C. with oscillation for 24 hours;

Step 2, centrifugal concentration: the cultured microorganisms are separated by a high-speed centrifuge to get the microorganisms, the temperature of the centrifugal chamber is 4° C. , the rotating speed is 4000 rpm, and the duration is 15 min; after centrifugation, the supernatant is removed, and the precipitate is dissolved in the fresh culture solution, the volume of the fresh culture solution is 1/10 of the original volume, that is, the 10 L microorganism solution is concentrated into 1 L, and the concentrated microorganisms are filled into a plastic water bag and stored at 4° C.;

Step 3, low-temperature transportation: the concentrated microorganisms are transported to the site in an incubator, and ice bags should be placed in the incubator to maintain the set low temperature in the incubator during transportation, and to ensure the rapid completion of the whole transportation process; after the microorganisms are transported to the site, they are immediately put into a refrigerator and stored at 4° C.;

Step 4, on-site expanded culture: the culture medium used for expanded culture comprises: industrial soybean peptone 25 g/L, urea 10 g/L, MnSO₄ 12 mg/L, NiCl.6H₂O 24 mg/L; the pH value of the culture medium was adjusted to 9.0-10.0 with NaOH, and the culture time was 12 h; after the culture, the bacterial activity was tested by conductivity method;

The cultured microorganisms were diluted with 0.9% NaCl solution which is cementing solution, and immediately used for on-site foundation reinforcement after dilution; the dilution ratio is 2:1; the solution could also be diluted with seawater nearby, and the dilution ratio was 3:1.

The said heavy hammer has a diameter of 200 mm-600 mm, a length of 1 m-5 m and a weight of 1.5-3.5 tons, the plate compactor is a 15-ton rammer composed of steel plates, the bottom surface of the rammer is round, the diameter of the hammer bottom is 2 m, and two exhaust holes with a diameter of 300 mm are arranged in the rammer.

The technical principle of the invention is as follows:

Firstly, the soil between piles is compacted twice. First of all, the soil between piles and the pile body are compacted by gravitational potential energy. The technical hole-forming method of the present invention features in punching and cutting the foundation soil to form a hole in way of free-falling body with a 3.5-ton heavy hammer, because soil is not taken in the construction process, the soil in the pile casing area is squeezed to the surrounding foundation soil, the soil body is compacted, the pores of the surrounding foundation soil are reduced, the compactness and bearing capacity of the foundation soil are improved, and the first compaction is finished; after the hole reaches the design elevation, lift the heavy hammer again to tamp the filler; due to the limited constraint of the surrounding foundation soil, the diameter of the pile will be larger than that of the hole; therefore, during the pile forming process, part of the foundation soil of the pile body will be squeezed around out again, forming the second compaction of the foundation soil around the pile.

Secondly, equal energy control is used in the construction process. In the whole construction process, the same column hammer is used, lifting it in way of free falling body with the same height to tamp the tilling material, and through the same one-stroke penetration extent as a control index, the measurement of the last stroke of heavy hammer compaction is similar to a super-large dynamic cone penetration test, so the same one-stroke penetration extent indicates that the compactness of pile body and surrounding foundation soil is basically consistent. By controlling the filling quantity and the last blow penetration extent, the original uneven foundation becomes uniform, which will help control the uneven settlement.

Thirdly, adding a filler composed of aggregate and microbial solidification liquid, under the ramming of the heavy hammer, the coarse aggregate and fine aggregate in the aggregate are fully compacted, and the microbial solidification solution is fully mixed with the aggregate; microorganisms continuously cement aggregate and its surrounding strata in the process of metabolism, which greatly improved the strength of single pile in the later period; the high temperature generated by hammering is beneficial to the growth of microorganisms, thus beneficial to controlling uneven settlement.

The invention has the beneficial effects as follows:

The composite foundation provided by the present invention with great bearing capacity is highly useful; the construction machinery is simple to operate, easy to move; the construction processes are easy to implement, and has a high construction quality assurance rate; the construction is efficient, short and quick; in the construction that produces no mud, the filling materials are coral aggregate or construction waste, which are obtained locally; this invention complies with the concept of green development by turning waste into wealth, protecting environment and reducing project cost.

### BRIEF DESCRIPTION OF THE FIGURES

FIG. 1 is a schematic view of the overall structure of the said composite foundation according to the present invention.

FIG. 2 is a schematic view of the construction principle of the said composite foundation according to the present invention.

FIG. 3 is a process flow diagram of the said composite foundation construction method according to the present invention.

The annotations in the above figure are as follows: 1. Pile body; 2, cushion layer; 3, aggregate; 4, weight hammer; 5, microbial solidification liquid.

### DETAILED DESCRIPTION OF THE INVENTION

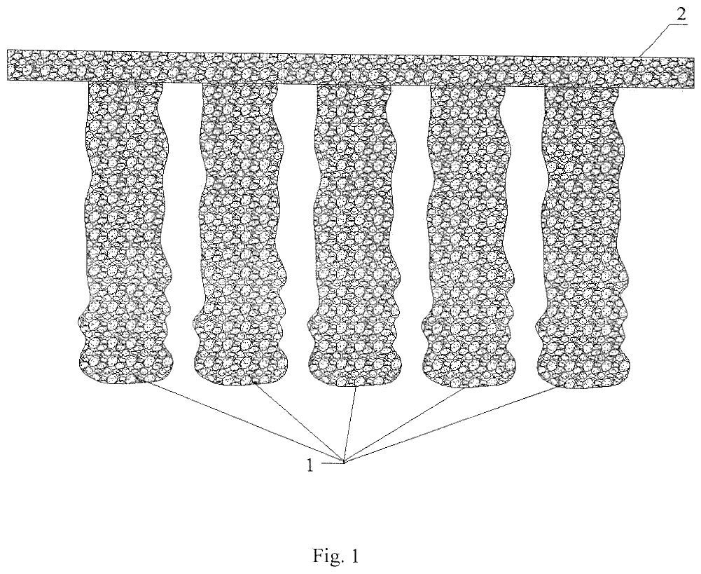

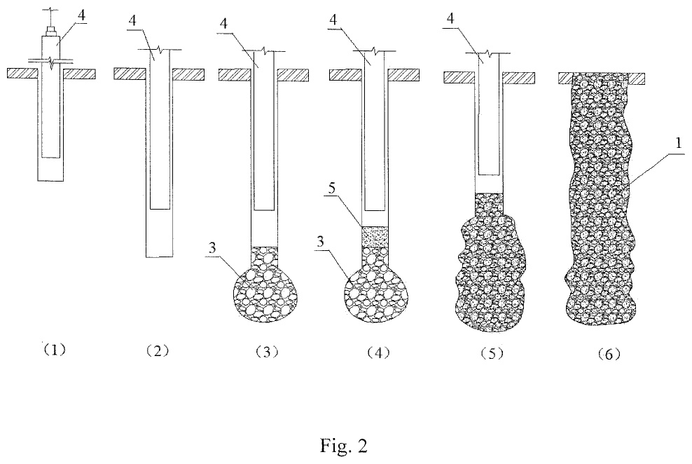

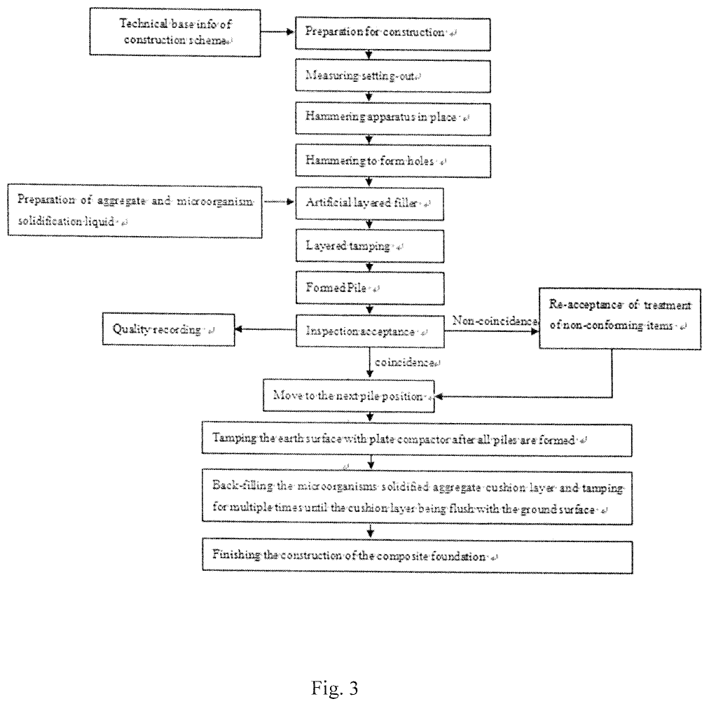

See FIG. 1 through FIG. 3:

The equal energy deformation composite foundation using microorganism to solidify aggregate provided by the present invention comprises a pile body 1 and a cushion layer 2, wherein a plurality of pile 1 are arranged in the pile body, the cushion layer 2 is arranged at the top of the pile body 1, the pile body I is connected into a whole structure by the cushion layer 2, and a aggregate 3 solidified by microorganism is filled in the pile body 1 and the cushion layer 2.

The microorganism is Bacillus pasturii, purchased from the German Collection of Microorganisms and Cell Cultures, and the strain number is DSM33

The aggregate 3 is coral aggregate or construction waste, the coral aggregate is composed of coarse aggregate and fine aggregate, the coarse aggregate is coral gravel, and the fine aggregate is coral sand, namely calcareous sand; construction waste includes concrete block, crushed stone, plain soil, metal, brick, tile and gypsum; after treatment of screening, rolling and crushing, the particle size of construction waste is ≤30 mm.

A construction method of equal energy deformation composite foundation using microorganism to solidify aggregate provided by the present invention, the method is as follows:

Step 1, cleaning and leveling the site;

Step 2, construction preparation: carrying out construction setting-out and line inspection; checking and adjusting construction equipment;

Step 3, the pile driver in place: the center of a heavy hammer 4 is aligned with the center of the pile position;

Step 4, forming a hole by hammering: lifting the heavy hammer 4 at a certain height to make it fall freely and impact the foundation soil to form a hole to a design or controlled depth;

Step 5, filling the aggregate 3 into the hole, pouring a microbial solidification liquid 5 with the same volume as the aggregate 3, wherein the microbial solidification liquid 5 is mainly composed of bacteria liquid and cementing liquid, lifting the heavy hammer 4 at a certain height, and ramming the filler repeatedly;

Step 6, under the action of standard ramming energy, the last penetration amount of the heavy hammer 4 is measured, and when it is no greater than the design requirement, the work of step 5 is repeated;

Step 7, repeating the steps 5 and the step 6, ramming and filling the pile hole to the ground, and finally forming an energy deformation compaction pile using microorganism to solidify aggregate in the foundation;

Step 8, after one pile is formed, the equipment is moved to the next pile;

Step 9, after all piles are formed, the ground of the construction area is tamped by using a plate compactor;

Step 10, back-filling a layer of aggregate 3 and the microbial solidification liquid 5 with the same volume as the aggregate 3 on the tamped ground, the back-filling elevation is higher than the ground surface by more than 0.2 m, then lifting the plate compactor to a certain height, and ramming the aggregate cushion after microbial solidification on the ground repeatedly until the cushion is flush with the ground surface.

The aggregate 3 is coral aggregate or construction waste, the coral aggregate is composed of coarse aggregate and fine aggregate, the coarse aggregate is coral gravel, and the fine aggregate is coral sand, namely calcareous sand; construction waste includes concrete block, crushed stone, plain soil, metal, brick, tile and gypsum; after treatment of screening, rolling and crushing, the particle size of construction waste is 30 mm.

The microorganism is Bacillus pasturii, purchased from the German Collection of Microorganisms and Cell Cultures, and the strain number is DSM33; the microorganism solution is obtained by means of indoor sterile culture, centrifugal concentration, low temperature transportation and on-site expanded culture, the specific method is as follows:

Step 1, indoor cultivation: every liter of culture medium contains tryptone 15.0 g, Soybean peptone 5.0 g, sodium chloride 5.0 g, putting the prepared nutrient solution in an autoclave, autoclaving at 121° C. for 20 min, and then cool it down in a sterile operation table; in order to avoid the decomposition of urea at high temperature, 20 g urea is added into the bottle when the temperature drops to room temperature, and the pH is adjusted to 7.3; after microbial inoculation, it is incubated at constantly 30° C. with oscillation for 24 hours;

Step 2, centrifugal concentration: the cultured microorganisms arc separated by a high-speed centrifuge to get the microorganisms, the temperature of the centrifugal chamber is 4° C. , the rotating speed is 4000 rpm, and the duration is 15 min; after centrifugation, the supernatant is removed, and the precipitate is dissolved in the fresh culture solution, the volume of the fresh culture solution is 1/10 of the original volume, that is, the 10 L microorganism solution is concentrated into 1 L, and the concentrated microorganisms are filled into a plastic water bag and stored at 4° C.;

Step 3, low-temperature transportation: the concentrated microorganisms are transported to the site in an incubator, and ice bags should be placed in the incubator to maintain the set low temperature in the incubator during transportation, and to ensure the rapid completion of the whole transportation process; after the microorganisms are transported to the site, they are immediately put into a refrigerator and stored at 4° C.;

Step 4, on-site expanded culture: the culture medium used for expanded culture comprises: industrial soybean peptone 25 g/L, urea 10 g/L, MnSO₄ 12 mg/L, NiCl.6H₂O 24 mg/L; the pH value of the culture medium was adjusted to 9.0-10.0 with NaOH, and the culture time was 12 h; after the culture, the bacterial activity was tested by conductivity method;

The cultured microorganisms were diluted with 0.9% NaCl solution which is cementing solution, and immediately used for on-site foundation reinforcement after dilution; the dilution ratio is 2:1; the solution could also be diluted with seawater nearby, and the dilution ratio was 3:1.

The heavy hammer 4 has a diameter of 200 mm-600 mm, a length of 1 m-5 m and a weight of 1.5-3.5 tons, the plate compactor is a 15-ton rammer composed of steel plates, the bottom surface of the rammer is round, the diameter of the hammer bottom is 2 m, and two exhaust holes with a diameter of 300 mm are arranged in the rammer.

The technical principle of the invention is as follows:

Firstly, the soil between piles is compacted twice. First of all, the soil between piles and the pile body are compacted by gravitational potential energy. The technical hole-forming method of the present invention features in punching and cutting the foundation soil to form a hole in way of free-falling body with the 3.5-ton heavy hammer 4, because soil is not taken in the construction process, the soil in the pile casing area is squeezed to the surrounding foundation soil, the soil body is compacted, the pores of the surrounding foundation soil are reduced, the compactness and bearing capacity of the foundation soil are improved, and the first compaction is finished; after the hole reaches the design elevation, lift the heavy hammer 4 again to tamp the filler; due to the limited constraint of the surrounding foundation soil, the diameter of the pile will be larger than that of the hole; therefore, during the pile forming process, part of the foundation soil of the pile body will be squeezed around out again, forming the second compaction of the foundation soil around the pile.

Secondly, equal energy control is used in the construction process. In the whole construction process, the same column hammer is used, lifting it in way of free falling body with the same height to tamp the filling material, and through the same one-stroke penetration extent as a control index, the measurement of the last stroke of heavy hammer 4 compaction is similar to a super-large dynamic cone penetration test, so the same one-stroke penetration extent indicates that the compactness of pile body and surrounding foundation soil is basically consistent. By controlling the filling quantity and the last blow penetration extent, the original uneven foundation becomes uniform, which will help control the uneven settlement.

Thirdly, adding a filler composed of aggregate 3 and microbial solidification liquid 5, under the ramming of the heavy hammer 4, the coarse aggregate and fine aggregate in the aggregate are fully compacted, and the microbial solidification solution 5 is fully mixed with the aggregate 3; microorganisms continuously cement the aggregate 3 and its surrounding strata in the process of metabolism, which greatly improved the strength of single pile in the later period; the high temperature generated by hammering is beneficial to the growth of microorganisms, thus beneficial to controlling uneven settlement.

1. An equal energy deformation composite foundation using a microorganism to solidify aggregate, the composite foundation comprising: a pile body and a cushion layer, wherein a plurality of piles are arranged in the pile body, the cushion layer is arranged at a top of the pile body, the pile body is connected into a whole structure by the cushion layer, and the aggregate solidified by the microorganism is filled in the pile body and the cushion layer.

2. The equal energy deformation composite foundation using microorganism to solidify aggregate according to claim 1, wherein the microorganism is Bacillus pasturii.

3. The equal energy deformation composite foundation using the microorganism to solidify aggregate according to claim 1, wherein the aggregate is a coral aggregate or a construction waste, the coral aggregate comprises a coarse aggregate and a fine aggregate, the coarse aggregate is coral gravel, and the fine aggregate is coral sand, namely calcareous sand; the construction waste comprises a concrete block, a crushed stone, a plain soil, a metal, brick, a tile and a gypsum; after treatment of screening, rolling and crushing, a particle size of construction waste is less than or equal to 30 mm.

4. A construction method for an equal energy deformation composite foundation using a microorganism to solidify an aggregate, the construction method comprising the steps of:
cleaning and leveling a site;
construction preparation of the site including: carrying out construction setting-out and line inspection; and checking and adjusting construction equipment;
putting a pile driver in place: and centering a heavy hammer of the pile driver with a center of a pile position;
forming a hole by hammering including lifting a heavy hammer at a certain height to make it fall freely and impact a foundation soil to form a hole to a controlled depth;
filling the aggregate into the hole, pouring a microbial solidification liquid with a same volume as the aggregate, wherein the microbial solidification liquid is composed of a bacteria liquid and a cementing liquid, lifting the heavy hammer to a certain height, and ramming a filler repeatedly;
ramming under the an action of standard ramming energy, wherein a last penetration amount of the heavy hammer is measured to a design requirement, and when a measurement is no greater than the design requirement, the filling the aggregate into the hole is repeated;
repeating the step of filling the aggregate into the hole and ramming under the action of standard ramming energy, then ramming and filling the hole to the ground, and forming an energy deformation compaction pile using the microorganism to solidify the aggregate in the foundation;
after one pile is formed, the construction equipment is moved to thea next pile;
after a plurality of piles are formed, a ground of the site is tamped by using a plate compactor;
back-filling a layer of aggregate and microbial solidification liquid with a same volume as an aggregate on the tamped ground, a back-filling elevation is higher than a ground surface by more than 0.2 m, then lifting the plate compactor to a certain height, and ramming the aggregate after microbial solidification on the ground repeatedly until the aggregate is flush with the ground surface.

5. The construction method of according to claim 4, wherein the aggregate is a coral aggregate or a construction waste, the coral aggregate comprises a coarse aggregate and a fine aggregate, the coarse aggregate is coral gravel, and the tine aggregate is coral sand; the construction waste comprises a concrete block, a crushed stone, a plain soil, a metal, brick, a tile and a gypsum; after a treatment of screening, rolling and crushing, the particle size of construction waste is less than or equal to 30 mm.

6. The construction method according to claim 4, wherein the microorganism is Bacillus pasturii and a microorganism solution is obtained by an indoor sterile culture, centrifugal concentration, low temperature transportation and an on-site expanded culture, the construction method further comprises the steps of:
putting a prepared nutrient solution comprising an indoor culture, where every liter of a culture medium contains tryptone 15.0 g, Soybean peptone 5.0 g, sodium chloride 5.0 g in an autoclave, autoclaving the indoor culture at 121° C. for 20 minutes, and then cool it the indoor culture down in a sterile operation table; in order to avoid the a decomposition of urea, 20 g of a urea is added into the a bottle when a temperature drops to room temperature, and a pH is adjusted to 7.3; after microbial inoculation, the indoor culture is incubated at constantly 30° C. with oscillation for 24 hours;
centrifugal concentration follows comprising: separating cultured microorganisms with a high-speed centrifuge to get the microorganisms, a temperature of a centrifugal chamber is 4° C., a rotating speed is 4000 rpm, and a duration is 15 min; after centrifugation, the a supernatant is removed, and the a precipitate is dissolved in fresh culture solution, a volume of the fresh culture solution is 1/10 of an original volume, that is, a 10 L microorganism solution is concentrated into a 1 L, and the concentrated microorganisms are filled into a plastic water bag and stored at 4° C.;
low-temperature transportation ensues whereby the concentrated microorganisms are transported to the a site in an incubator, and ice bags are placed in the incubator to maintain a set low temperature in the incubator during transportation, and to ensure a rapid completion of the low temperature transportation; after the microorganisms are transported to the site, they are immediately put into a refrigerator and stored at 4° C.;
on-site expanded culture follows whereby the culture medium used for expanded culture comprises: industrial soybean peptone 25 g/L, urea 10 g/L, MnSO₄ 12 mg/L, NiCl.6H₂O 24 mg/L; the a pH value of the culture medium is adjusted to 9.0-10.0 with NaOH, and a culture time is 12 h; after the a culture is formed, a bacterial activity was tested by conductivity;
the cultured microorganisms are diluted with a cementing solution which is 0.9% NaCl solution, and immediately used for on-site foundation reinforcement after dilution; a dilution ratio is 2:1; or the solution is diluted with seawater, and the dilution ratio was 3:1.

7. The construction method according to claim 4, wherein the heavy hammer has a diameter of 200 mm-600 mm, a length of 1 m-5 m and a weight of 1.5-3.5 tons, the plate compactor is a 15-ton rammer composed of steel plates, a bottom surface of the rammer is round, the a diameter of a bottom of the hammer is 2 m, and two exhaust holes with a diameter of 300 mm are arranged in the rammer.
